(19) United States
(12) Patent Application Publication
(10) Pub. No.: US 2024/0427816 A1
(43) Pub. Date: Dec. 26, 2024

(54) METHODS AND SYSTEMS FOR IMPLEMENTING MULTI-CHANNEL SERVICE PLATFORMS OVER AUDIO-BASED COMMUNICATION CHANNELS

(71) Applicant: Polyview Health, Inc., San Diego, CA (US)

(72) Inventors: Jason Hwang, Henderson, NV (US); Gregory Deocampo, Safety Harbor, FL (US); Dimitri Arges, Henderson, NV (US)

(21) Appl. No.: 18/748,175

(22) Filed: Jun. 20, 2024

Related U.S. Application Data
(60) Provisional application No. 63/509,910, filed on Jun. 23, 2023.
(60) Provisional application No. 63/509,973, filed on Jun. 23, 2023.
(60) Provisional application No. 63/510,006, filed on Jun. 23, 2023.
(60) Provisional application No. 63/510,019, filed on Jun. 23, 2023.
(60) Provisional application No. 63/510,608, filed on Jun. 27, 2023.
(60) Provisional application No. 63/604,930, filed on Dec. 1, 2023.

(51) Int. Cl. G06F 16/635; G06F 40/58

(52) U.S. Cl. CPC G06F 16/635; G06F 40/58

(57) ABSTRACT

Methods and systems are described herein for implementing a multi-channel service platform that facilitates communication sessions between one or more user devices on an audio platform. The method may comprise intercepting a first audio segment over an audio channel of a communication session between a first user device and a second user device. The audio segment is transmitted by the second user device. The method may further comprise identifying user profile data associated with the first user device, and generating, based on the user profile data, a second audio segment using a machine-learning model. The second audio segment is contextually related to the first audio segment. The method may further comprise transmitting, to the first user device, the second audio segment over the audio channel of the communication session. When received by the first user device, the second audio segment is presented over a portion of the first audio segment.

### CROSS-REFERENCE TO RELATED APPLICATIONS

The present patent application claims the benefit of priority to U.S. Provisional Patent Applications 63/509,910, 63/509,973, 63/510,006, and 63/510,019, all of which were filed Jun. 23, 2023; U.S. Provisional Patent Application 63/510,608, filed Jun. 27, 2023; and U.S. Provisional Patent Application 63/604,930, filed Dec. 1, 2023, which are all incorporated herein by reference in their entirety for all purposes.

### TECHNICAL FIELD

This disclosure relates generally to facilitating communication sessions, and more specifically to implementing a multi-channel service platform that facilitates communication sessions between one or more user devices over an audio communication channel.

### BACKGROUND

Telehealth can be conducted between a patient and a healthcare provider over a variety of communication channels. These telehealth calls are often facilitated in a standard format (e.g., a phone call performed over a cellular network). Because of the basic nature of current telehealth calls, the calls may not be as beneficial or as efficient as they could be for both the patient and the healthcare provider. For example, it might be more difficult for a patient or healthcare provider provide relevant information in an understandable form or format. In addition, telehealth calls often cause the patient to feel disengaged from the appointment and the healthcare provider, which may create an environment where the patient might not trust the healthcare provider's advice or might not feel comfortable asking questions.

### SUMMARY

Methods and systems are described herein for implementing a multi-channel service platform that facilitates communication sessions between one or more user devices on an audio platform. The method comprises: intercepting a first audio segment over an audio channel of a communication session between a first user device and a second user device, where the audio segment is transmitted by the second user device, identifying user profile data associated with the first user device, generating, based on the user profile data, a second audio segment using a machine-learning model, where the second audio segment is contextually related to the first audio segment, and transmitting, to the first user device, the second audio segment over the audio channel of the communication session, where when received by the first user device, the second audio segment is presented over a portion of the first audio segment.

Systems are described herein for implementing a multi-channel service platform that facilitates communication sessions between one or more user devices on an audio platform. The systems include one or more processors and a non-transitory computer-readable storage medium storing instructions that, when executing by the one or more processors, cause the one or more processors to perform any of the methods as previously described.

A non-transitory computer-readable medium described herein may store instructions which, when executed by one or more processors, cause the one or more processors to perform any of the methods as previously described.

These illustrative examples are mentioned not to limit or define the disclosure, but to aid understanding thereof. Additional embodiments are discussed in the Detailed Description, and further description is provided there.

### BRIEF DESCRIPTION OF THE DRAWINGS

Features, instances, and advantages of the present disclosure are better understood when the following Detailed Description is read with reference to the accompanying drawings.

FIG. 1 illustrates a block diagram of example communication network configured to facilitate secured communication sessions between users according to aspects of the present disclosure.

FIG. 2 illustrates a flowchart of an example process for a first user device to initialize an administrative session according to aspects of the present disclosure.

FIG. 3 illustrates a flowchart of an example process for initializing settings for a communication session according to audio input from the first user device according to aspects of the present disclosure.

FIG. 4 illustrates a flowchart of an example process for facilitating a communication session between one or more user device on an audio communication platform.

FIG. 5 illustrates an example computing device according to aspects of the present disclosure.

### DETAILED DESCRIPTION

Various instances of the disclosure are discussed in detail below. While specific implementations are discussed, it should be understood that this is done for illustration purposes only. A person skilled in the relevant art will recognize that other components and configurations may be used without parting from the spirit and scope of the disclosure.

Methods and systems are described herein for providing a communication session environment in an audio communication platform within a multi-channel service platform. In some instances, a communication session may be conducted between a first user device associated with a patient (e.g., such as a current or new patient, etc.) and a second user device associated with a healthcare provider (e.g., such as a doctor, nurse, therapist, administrator, and/or other healthcare professional) as part of a telehealth session. One or more other user devices may also be connected to the communication session. The one or more other user devices may be associated with other users (e.g., such as additional patients, users associated with the patient such as a nurse, additional healthcare providers, etc.). For example, an adult child of an elderly parent may participate in a communication session with the elderly parent and a healthcare provider of the elderly parent.

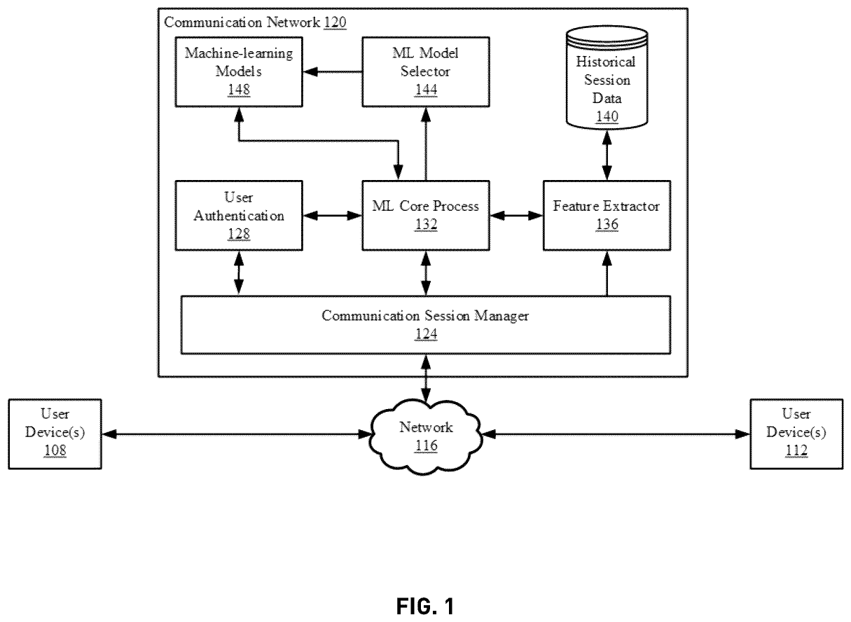

The first user device may transmit a communication session request to a communication network (e.g., communication network 120 of FIG. 1, an application, network, etc.) using an application executing on the user device or another device, using webpage or web application, using an audio-based communication channel, etc. The communication session may be facilitated over one or more communication channels (e.g., telephone or other audio-based communication channels). The communication session request may include a set of communication session parameters usable to define a new communication session. When using an audio-based communication channel, the communication session parameters may be defined based on touch-tone commands or by a natural-language understanding machine-learning model configured to identify parameters from natural language speech.

The set of communication session parameters may include, but are not limited to, a quantity of user devices that are authorized to connect to the communication session, an identification of the user devices or the users thereof (e.g., such a device identifier, Internet Protocol address, email address, phone number, username, a user identifier, combinations thereof, or the like), a length of the communication session, an identification of one or more communication channels authorized for the communication session (e.g., such as, audio, text messaging, email, instant messaging, combinations thereof, or the like), sound settings, microphone settings, collaborative environment parameters, privacy and/or encryption parameters, artificial intelligence accessibility, date and time, etc. The set of communication session parameters may be modified at any time prior to and/or during the communication session.

In an illustrative example, the set of communication session parameters may include an identification of one or more other user devices, which may include the second user device. The one or more other user devices may be associated with a healthcare provider, another patient, someone associated with the patient (e.g., such as a social worker, nurse, aide, etc., a third party, etc.). The first user device may identify the one or more other user devices as user devices to be invited to connect to the communication session. In some examples, the communication session request may not include an identification of one or more other user devices. For example, the first user device may initiate a communication session with a chatbot (e.g., a machine-learning algorithm with audio output capabilities) to change one or more settings associated with a user profile, to present a non-urgent question, to perform administrative tasks, any combination thereof, or the like.

The communication network may transmit a notification to the one or more other user devices invited to the communication session using information of the set of communication session parameters. The notification may be transmitted over a same or different communication channel as the communication channel identified by the communication session parameters. The notification may include a representation of one or more communication session parameters of the set of communication session parameters. The one or more other user devices may accept or decline the communication session. A user device of the one or more second user devices may request a modification to one or more of the communication session parameters (e.g., such the date/time of the communication session, identification of user devices authorized to access the communication session, etc.). The communication network may receive a response from the one or more other user devices (or other user devices associated with the communication request). The one or more other user devices may modify a response of accepting or declining the communication session request (e.g., changing an acceptance to a decline, changing a decline to an acceptance, etc.). The communication session parameters may determine an action if no response is received from a user device of the one or more other user devices. In some examples, a response is not needed from one or more other user devices (e.g., the first user device requests a communication session with a chatbot) and the communication session may be automatically initiated and/or initiated at an indicated date/time.

In some examples, the communication network may facilitate access to a telehealth environment. The telehealth environment may be operated by the communication network, the healthcare provider, an entity associated with the healthcare provider, etc. In some instances, the telehealth environment may be an application, website, or like configured to store and provide information associated with the first user device and facilitate the communication session. In other instances, the telehealth environment may include an audio interface through which the one or more user devices may communicate or through which a user device may interact with the communication network. For example, the audio interface may operate in parallel to the audio-based connection to execute commands of the communication network (e.g., request information, execute functions, etc.) using spoken keywords, touch tones, spoken natural language communications, etc.

The telehealth environment may include a post-session environment. The post-session environment may include an identification of previous communication sessions associated with the first user device. The telehealth environment may include an application or plugin configured to replay a previous communication session. In some instances, the telehealth environment may include an audio-operated menu configured to permit the first user device to navigate to the post-session environment using one or more audio commands (e.g., using one or more touch tones of a telephone, spoken commands, natural language communications, etc.). The post-session environment may store recordings of one or more prior communication sessions. For example, the first user device can access the recordings of the one or more prior communication sessions by stating, “review past sessions.” The telehealth environment may employ one or more machine-learning models (e.g., a machine-learning model configured to process natural language) to detect one or more commands from the first user device.

The post-session environment may also include resources presented by and/or discussed by user devices during communications sessions, resources provided by user devices before or after a communication session (e.g., charts, explanations, prescriptions, test results, notes, instructions, etc.), access to artificial intelligence (AI) resources (e.g., natural language processors for speech-to-text, text-to-speech, translation, classification, etc.; large language models or other generative models for automated communication and information generation; etc.), other resources, administrative links (e.g., pay bill, schedule appointment, etc.), transcripts of prior communication sessions, potential questions for the healthcare provider, etc. In some examples, the first user device may direct the audio interface to send one or more of the resources presented by and/or discussed by user devices during the communication session via a non-audio-based communication channel such as, but not limited to, text message, email, instant message, an application, a webservice or web application, a website, etc. For example, if a healthcare provider discusses a particular diagram of a knee with a patient during a communication session, the patient may request a copy of the diagram to be transmitted via email after the conclusion of the communication session.

The communication network may provide access to the communication session when a current time corresponds to a time identified by a communication session parameter. The communication network may establish the communication session through the telehealth environment using a sub-environment configured to enable communications over one or more communications channels identified by a communication session parameter of the set of communication session parameters. Alternatively, the communication network may establish the communication session using a third-party environment (e.g., audio conferencing application or environment provided by an entity other than the communication network, etc.). The first user device and the one or more other user devices may access the communication network and/or the communication session using via the Internet, local or wide area network, a cellular network, etc.

In some examples, the first user device and the second user device may dial a particular phone number, associated with the telehealth environment, at the time identified by the communication session parameter. The telehealth environment may receive input from the first user device and the second user device, such as a phone number, session identifier, token, cryptographic key, location, associated user, etc. The telehealth environment may utilize the input to redirect the first user device and second user device to an appropriate sub-environment facilitated by the telehealth environment. In some examples, the particular phone number may be presented to the first user device and the second user device specifically for the communication session. For example, upon scheduling the communication session, the first user device may receive an email which includes a phone number to call at the time of the communication session. In some examples, the first or second user device may select a link presented via an application, text message, website, email, any combination thereof, or the like, which causes the user device to dial the phone number of the communication session or connect to a network location corresponding to the communication session. In some examples, the first user device may dial a phone number associated with the healthcare provider. Upon dialing, the telehealth environment may detect the first user device is associated with an upcoming communication session and may redirect the first user device to an appropriate cellular network line.

In some examples, the communication network may include an access sequence restriction that defines an order in which user devices may access the communication session. The access sequence restriction may comprise an access sequence order, wherein the access sequence order may position the first user device and the one or more other user devices sequentially. The communication network may connect the one or more user devices positioned lower in the access sequence order to a temporary environment (e.g., a virtual waiting room, etc.) until the one or more user devices positioned higher in the access sequence order connect to the communication session. For example, the communication network may indicate that the first user device cannot join the communication session until the second user device connects to the communication session.

The temporary environment may enable user devices to access information associated with the communication session and transmit information to the communication network or healthcare provider (e.g., intake healthcare forms, medical history, insurance information, etc.). The information associated with the communication session can include a tutorial or other introductory information that provides information on features of the communication network and communication session as well as how to access the features, information associated with the healthcare provider (e.g., such as names and/or address of healthcare practitioners, services offered by the healthcare provider, branches of medicine practiced by the healthcare provider, affiliated healthcare providers, emergency information, etc.). The information associated with the communication session may be available in an audio format.

The temporary environment may include one or more automated services (e.g., chatbots, large language models, generative models, natural language understanding models, audio processors, etc.) configured to interact with the user devices of the temporary environment over various communication channels. The one or more automated services may be configured to communicate using natural language communications in a language selected by a user device (or detected from communications transmitted by the user device) communicating with the automated services. The user devices may ask questions related to the communication session or the purpose for the communication session, request information associated with the healthcare provider or communication network, etc. and receive responses from the automated service in a natural language format and in the selected language.

In some instances, the second user device may be presented with the questions or requests for information from the first user device. The communication network may be configured by the healthcare provider to direct an automated service to provide responses. Alternatively, the second user device may configure an automated service (e.g., stored on the communication network or on the second user device) to provide responses. Alternatively, or additionally, the healthcare provider may provide responses. The communication network may identify responses generated by an automated service different from responses generated by the healthcare provider to enable a user to determine an origin of a particular response.

In some instances, the resources available in the temporary environment may be determined by an associated health system. The associated health system may be the hospital system affiliated with the clinic, office, hospital, etc. where the healthcare provider conducts healthcare services to patients. For example, the resources available in the temporary environment may include an audio overview of the treatments offered at the hospital, operating hours of the clinic and/or hospital, a phone number associated with the associated health system, or any other resource that may be specific to the associated health system. The healthcare provider may also select the resources available in the temporary environment. As another example, if the healthcare provider receives information indicating that the patient has a sore throat, the healthcare provider may generate a symptom checker for the patient to complete within the temporary environment, provide diagnostic information associated with sore throats and possible causes, provide treatment information, etc.

The communication session may be facilitated by a communication session environment within the telehealth environment. The communication session environment may include an audio interface generated by the communication network to facilitate the communication session. The audio interface may be different for each user device or class of user devices connected to the communication session. For example, the second user device may have access to one or more audio commands that the first user device may not have access to. The audio interface may enable enhanced presentation of information associated with the communication session during the communication session. For example, the communication session parameters may enable one or more machine-learning algorithms to supplement audio segments with translations, definitions, or descriptions, etc. For example, if the healthcare provider uses a medical term, the one or more machine-learning algorithms may output a definition of the medical term to the patient via a second audio segment that may be presented after the current audio segment. The machine-learning algorithms may be configured to detect keywords or phrases to trigger the presentation of supplemental audio segments. Alternatively, the machine-learning algorithm may provide the supplemental audio segments in response to a command or request from a user device. The supplemental audio segments may be presented unilaterally such no other user device will hear the supplemental audio segments other than the requesting user device.

When the communication session terminates, additional resources may be available via the audio interface to the user devices connected to the communication session. The resources may be generated by one or more machine-learning models or may be provided by one or more of the user devices connected to the communication session. For example, the communication network may utilize a natural language processing (“NLP”) machine-learning model to generate a transcript of the communication session to a user device. The transcript can be downloaded (e.g., as a file, etc.) or presented to the requesting user device over the audio interface. As another example, if the healthcare provider mentions a specific scientific study in the communication session, the communication network may provide an access to the scientific study (e.g., presented using a text-to-speech machine-learning model via the audio interface, downloaded as a file, etc.). A user device, via the communication network, may request that the resources be transmitted to the user device via text message, email, instant message, any combination thereof, or the like. In some instances, the user device may initiate an administrative session, which may be a type of communication session that facilitates access to audio interface resources, historical communication sessions, records, etc. The user device may initiate an administrative session to complete one or more administrative tasks, like request the resources pertaining to a prior communication session, pay an outstanding bill, change one or more settings associated with the user device, request a new communication session, any combination thereof, or the like.

During the communication session, the communication network may utilize the communication (e.g., audio) between the user devices connected to the communication session and generate a data package containing a recording of the communication session (e.g., containing audio content outputted during the communication session) and data generated by other AI-assisted resources. After the communication session terminates, the user devices connected to the communication session may receive a notification (e.g., via email, telehealth environment notification, phone call, text message, etc.) indicating the data package is available for retrieval. The data package may be accessible to each user device but may differ based on the user device. For example, the data package of each user device may include a representation of the communication session that was presented to that user device during the communication session. The first user device may access a data package with a representation of the communication session relative to the first user device (e.g., including the audio transmitted from and received by the first user device during the communication session).

In some instances, the data package may include additional data derived from the communication session such as additional information about content presented during the communication session, statements made by the healthcare provider or patient, keywords or phrases, definitions or explanations related to particular keywords and/or phrases, annotations, timestamps associated with contextually relevant portions of the communication session, etc. The additional data may be generated automatically (e.g., by one or more of the AI resources or automated services previously described), generated by the healthcare provider, generated by the patient, or by another entity that was connected to the communication session.

FIG. 1 illustrates a block diagram of example communication network configured to facilitate secured communication sessions between users according to aspects of the present disclosure. Communication network 120 may facilitate communication sessions between a first user device operated by a patient (e.g., user device 108) and a second user device operated by a healthcare provider (e.g., user device 112). Communication network 120 may enable one or more other devices associated with the first user device (e.g., such as nurses, aides, social workers, parents, adult children, etc.) and/or the second user device (e.g., nurses, assistants, other doctors, administrators, etc.) to connect to the communication session.

Communication network 120 may include one or more processing devices (e.g., computing devices, mobile devices, servers, databases, etc.) configured to operate together to provide the services of communication network 120. The one or more processing devices may operate within a same local network (e.g., such as a local area network, wide area network, mesh network, etc.) or may be distributed processing devices (e.g., such a cloud network, distributed processing network, or the like). User device 108 and user device 112 may connect to communication network 120 directly or through one or more intermediary networks 116 (e.g., such as the Internet, virtual private networks, telephone networks, etc.).

The first user device or the second user device may request a new communication session using communication session manager 124. The request may include parameters such as user profile data (associated with as user of the first user device or the second user device=), a purpose for establishing the communication session, a start time of the communication session, an expected duration of the communication session, settings of the communication session (e.g., audio channel settings, video channel settings, collaborative window settings, wrapper settings, etc.), combinations thereof, or the like.

In some instances, the first user device and/or the second user device may include with hardware and/or software permitting peer-to-peer facilitation of the communication session. For example, an application within the first user device may instantiate the communication session without accessing a central network, such as communication network 120. The hardware and/or software within the first user device and the second user device may facilitate any and/or all functions of communication network 120 described herein, including, but not limited to, communication session manager 124, ML core process 132, user authentication 128, any combination thereof, or the like.

Communication session manager 124 may instantiate a new communication session for the first user device and/or the second user device based on the request. The new communication session may include one or more environments to facilitate communications between the user devices connected to the communication session. The environment may include user interfaces, wrappers, resources, audio interfaces, application programming interfaces, etc. configured to extend the functionality of the communication session. The communication session manager 124 using ML core process 132 may provision one or more machine-learning models to enable any of the extended functionality. The one or more machine-learning models may be configured to provide natural language processing (e.g., using a large language model, bi-directional transformers, zero/few shot learners, deep neural networks, etc.), content generation (e.g., using large language models, deep neural networks, generative adversarial networks, etc.), single variate or multivariate classifiers (e.g., k-nearest neighbors, random forest, logarithmic regression, decision trees, support vector machines, gradient decent, etc.), image processing (e.g., using deep neural networks, convolutional neural networks, etc.), sequenced data processors (e.g., such as recurrent neural networks, etc. capable of processing datasets organized according to a taxonomic sequence), and/or the like. One or more of the one or more machine-learning models may be configured to process natural language communications (e.g., such as gestures, verbal, textual, etc.) to provide real-time translations and/or transcriptions, generate natural language communication capable of autonomous communication (e.g., a communication bot) or content generation (e.g., such generating natural language responses to requests for information, etc.), perform user authentication (e.g., to ensure users connected to the communication session are authorized to do so), and/or the like.

Communication session manager 124 may authenticate each user device that connects to the new communication session using user authentication 128. User authentication 128 may ensure that the user of a connected user device corresponds to an authorized user. To avoid exposing personal identifiable information or medical information, user authentication 128 may compare abstracted features associated with the user to corresponding abstracted features associated with an authorized user. In some instances, the abstracted features may include an abstracted representation of a username, password, token, public/private key, and/or the like. Communication session manager 124 may distribute passwords, tokens, public/private keys, and/or the like with an invitation to connect to the new communication session. Features may be abstracted using an abstraction function (e.g., such as a hash function, cryptographic function, etc.) and then distribute passwords, tokens, public/private keys, and/or the like of the features.

In some instances, user authentication 124 may obtain one or more audio segments received from a user device to be authenticated. The audio segments may include a representation of a voice of the user. User authentication 124 may transmit a request to machine-learning (ML) core process 132 to process the audio. For example, using a first machine-learning model, user authentication 148 may process audio segments including a representation of the user's voice. The second machine-learning model may process the audio segments to derive abstracted features associated with the audio segments, the second machine-learning model may be configured to identify pitch, tone, speech velocity, pause frequency and length, diction, accent, language, etc. of the audio segment represented as a sequence of numerical values. The abstracted features can be compared to historical abstracted features of an authenticated user to determine if the user associated with the abstracted features is the same user as the user associated with the historical abstracted features.

Communication session manager 124 may pass communications extracted over the communication session to ML core process 132 to process the communications using the one or more machine-learning models. ML core process 132 may monitor one or more machine-learning models configured to provide the services of the communication network. ML Core process 132 may train new machine-learning models, retrain (or reinforce) existing machine-learning models, delete machine-learning models, and/or the like. Since ML Core process 132 manages the operations of a variety of machine-learning models, each request to ML core process 132 may include an identification of a particular machine-learning model, a requested output, or the like to enable ML core process 132 to route the request to an appropriate machine-learning model or instantiate and train a new machine-learning model. Alternately, ML core process 132 may analyze data to be processed that is included in the request to select an appropriate machine-learning model configured to process data of that type.

If ML core process 132 cannot identify a trained machine-learning model configured to process the request, then ML core process 132 may instantiate and train one or more machine-learning models configured to process the request. Machine-learning models may be trained to process a particular input and/or generate a particular output. ML core process 132 may instantiate and train machine-learning models based on the particular data to be processed and/or the particular output requested. For example, user sentiment analysis (e.g., user intent, etc.) may be determined using a natural language processor and/or a classifier while image processing may be performed using a convolutional neural network.

ML core process 132 may select one or more machine-learning models based on characteristics of the data to be processed and/or the output expected. ML core process 132 may then use feature extractor 136 to generate training datasets for the new machine-learning models (e.g., other than those models configured to perform feature extraction such as some deep learning networks, etc.). Feature extractor 136 may define training dataset using historical session data 140. Historical session data 140 may store features from previous communication sessions. In some instances, the previous communication sessions may not involve the user of the first user device or the user of the second user device. Previous communication sessions may include manually and/or procedurally generated data generated for use in training machine-learning models. Historical session data 140 may not store any information associated with healthcare providers or patients. Alternatively, historical session data 140 may store features extracted from communication session involving the user of the first user device, the user of the second user device, and/or other patients and/or other healthcare providers.

Feature extractor 136 may extract features based on the type of model to be trained and the type of training to be performed (e.g., supervised, unsupervised, etc.) from historical session data 140. Feature extractor 136 may include a search function (e.g., such as procedural search, Boolean search, natural language search, large language model assisted search, or the like) to enable ML core process 132, an administrator, or the like to search for particular datasets within historical session data 140 to improve the data selection for the training datasets. Feature extractor 136 may aggregate the extracted features into one or more training datasets usable to train a respective machine-learning model of the one or more machine-learning models. The training datasets may include training datasets for training the machine-learning models, training datasets to validate an in-training or trained machine-learning model, training datasets to test a trained machine-learning model, and/or the like. The one or more training datasets may be passed to ML core process 132, which may manage the training process.

Feature extractor 136 may pass the one or more training datasets to ML core process 132 and ML core process 132 may initiate a training phase for the one or more machine-learning models. The one or more machine-learning models may be trained using supervised learning, unsupervised learning, self-supervised learning, or the like. The one or more machine-learning models may be trained for a predetermined time interval, a predetermined quantity of iterations, until one or more target accuracy metrics have exceeded a corresponding threshold function (e.g., accuracy, precision, area under the curve, logarithmic loss, F1 score, weighted human disagreement rate, cross entropy, mean absolute error, mean square error, etc.), user input, combinations thereof, or the like. Once trained, ML core process 132 may validate and/or test the trained machine-learning models using additional training datasets. The machine-learning models may also be trained at runtime using reinforcement learning.

Once the machine-learning models are trained, ML core process 132 may manage the operation of the one or more machine-learning models (stored with other machine-learning models in machine-learning models 148) during runtime. ML core process 132 may direct feature extractor 136 to define feature vectors from received data (e.g., audio segments from the first user device or the second user device). In some instances, ML core process 132 may facilitate generation of a feature vector each time there is a change in the communication channel (e.g., an audio segment is transmitted over the communication channel), a timestamp relative to a start time of the communication session, or the like. ML core process 132 may continually execute the one or more machine-learning models to generate corresponding outputs. ML core process 132 may evaluate the outputs to determine whether to manipulate a user interface of the communication session based on the output (e.g., initiate an automated conversation with a bot, provide information associated with keywords spoken during the communication session, provide modifications to one or more outputted audio segments, etc.).

For example, ML core process 120 may detect a new audio segment over the communication session. ML core process 120 may execute a machine-learning model (e.g., such as a recurrent neural network, etc.) to process the audio segment to identify the words within the audio segment (if any) and a sentiment (e.g., a predicted meaning of the individual words or the words as a whole). ML core process 120 may execute another machine-learning model (e.g., such as a classifier, a large language model and/or transformer, a generative adversarial network, etc.) to generate content corresponding to the words and/or sentiment that can be provided to a user device. For instance, the words may include “My knee is swollen and painful” with a sentiment of “symptoms.” The other machine-learning model may process the words and sentiment to generate content for patient interface 200 such as information about ailments associated with knee pain and knee swelling, home treatments that may alleviate symptoms and/or improve mobility, possible questions that can be asked to the healthcare provider, etc. ML core process 120 may also use the other machine-learning model to generate content for provider interface such as symptoms, suggested follow up questions regarding the degree of swelling or the intensity of the pain, links for additional information associated with knee pain or knee swelling, links associated with ailments associated with knee pain or knee swelling, etc.

ML core process 120 may also detect commands within communications that can be facilitated by the communication network. For instance, a user device connected to the communication session may request information (e.g., associated with a disease, treatment, prescription, referral, or any other aspect of the communication session), content within an electronic medical record (EHR) such as previous communication session notes or test results, an identification of a referral (e.g., such as a name, practice area, contact information, etc.), combinations thereof, or the like. The audio segment that includes a detected command may be suppressed from distribution to user devices other than the user device that provided the command (e.g., using bandpass filters, a machine-learning model, etc.). The response to the command (e.g., any content generated or to be provided to satisfy the command) may be transmitted to just the user device that provided the command. The command may include keywords or natural language. In some instances, to avoid false positives, ML core process 120 may be configured to detect a wake word (e.g., such as an identifier of the machine-learning model or process configured to process the commands). Upon detecting the wake word, ML core process 120 may process the subsequent audio segment for a command. ML core process 120 may also prevent the subsequent audio segment from be transmitted to other user devices of the communication session. Commands available to user devices may be based on a role assigned to the user device. For example, user devices associated with patients may have access to different commands than user devices operated by healthcare providers.

ML core process may direct feature extractor 136 to define other feature vectors to process other data using machine-learning models of machine-learning models 148 in parallel with the aforementioned machine-learning models to provide other resources of communication network 120. ML core process 132 may execute any number of machine-learning models in parallel to provide the functionality of a communication session.

Communication session manager 124 may process received audio content into a format that can be interpreted by other user devices connected to the communication session. Communication session manager 124 may detect aa language spoken by a particular user device and if it is different from a language spoken by another user device, communication session manager 124 may automatically translate communications transmitted by the other user device into the language spoken by the particular user device and communications transmitted by the particular user device may be translated into the language spoken by the other user device. Communication session manager 124 may also receive outputs from machine-learning models 148 via ML core process 132 and determine whether to update the audio interfaces using the output and how to update the interfaces using the output (e.g., what volume to output a modified audio segment, what language to output the modified audio segment, to supplement the healthcare provider's diagnosis with additional information, etc.). Communication session manager 124 may continuously update the audio interfaces to present a dynamic, collaborative interface to each user device through the communication session. When the communication session terminates, communication session manager 124 may store the interfaces with an identifier associated with the communication session for further processing and/or replay by a user device of the communication session.

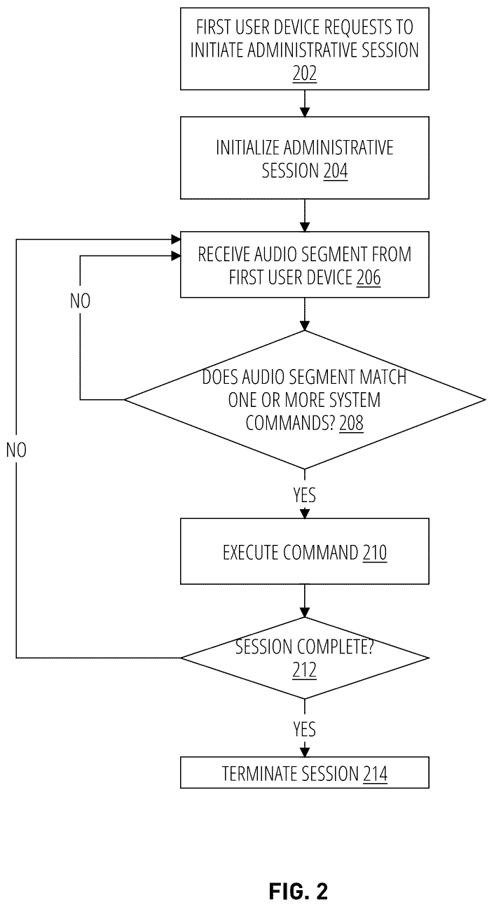

FIG. 2 illustrates a flowchart of an example process for a first user device to initialize an administrative session according to aspects of the present disclosure. Although the example process depicts a particular sequence of operations, the sequence may be altered without departing from the scope of the present disclosure. For example, some of the operations depicted may be performed in parallel or in a different sequence that does not materially affect that function of the example process. In other examples, different components of an example device or system that implements the example process may perform functions at substantially the same time or in a specific sequence.

At block 202, a first user device may request to initiate an administrative session. The first user device may be a user device operated by a patient. The user device may be a cell phone, smart phone, landline, computer, any combination thereof, or the like. An administrative session may be a communication session between the first user device and the communication network that facilitates access to functions of the communication network by the first user device without other user devices being connected to the same session. For example, an administrative session may be initiated to execute administrative tasks, such as paying a bill, provide and/or verifying insurance information, updating contact information of a patient, any combination thereof, or the like.

A communication manager may be instantiated to operate as an interface of the communication network. The communication manager may include one or more applications and/or processes that can be executed by the first user device and/or the communication network. In some instances, the communication manager may be configured to provide some of communication-assist features provided by the communication network (e.g., such as real-time translation and/or transcription, natural-language processing, natural language content generation (such as definitions, information, etc.), combinations thereof, or the like. The communication manager may also execute commands of the communication interface.

In some instances, the communication manager may facilitate execution of one or more machine-learning models of the communication network. The communication network may include one or more natural language machine-learning models (e.g., such as a large language model, bi-directional transformers, zero/few shot learners, deep neural networks, classifiers, etc.). The one or more machine-learning models may be configured to process natural language communications (e.g., speech, inflection, language, etc.), classify a context and/or intent, and provide responses to the first user device. The responses may be audio-based communication (e.g., an answer to a question, etc.) or execution of a command or function (e.g., such as updating an electronic health record, providing insurance information, etc.).

Alternatively, or additionally, communication manager may include the one or more machine-learning models. For example, when the communication manager is configured to operate as a local application or process of the first user device (e.g., executed by the first user device), the communication manager may execute to provide some or all of the services. For some services, the communication network may connect to the communication network (e.g., to access records of the communication network and execute commands, etc.). For other services, such as real-time translation, the communication manager may not connect to the communication network. For example, the communication manager may intercept communications received by the first user device over the audio-based connection and use the one or more machine-learning models to detect a language of the communication and translate the language into a language of the user of the first user device. The communication manager may present the translated communication in place of the original communication (e.g., such that the user of the first user device only hears the translated communications) or the communication manager may present the translated communication over (e.g., at higher volume or frequency) the original communication or after the original communication.

The communication manager may be configured to modify the audio-based connection based on communications received from the first user device. For example, the communication manager may detect languages spoken by the user of the first user device and cause communications transmitted to the first user device to be translated into a detected language. The communication manager may update the translation language based on subsequent communications transmitted by the first user device. For example, if the user of the first user device switches from English to Spanish, then the communication manager may cause the translation service to translate subsequent non-Spanish communications to be transmitted to the first user device into Spanish. The communication manager may modify characteristics of the audio-based connection (e.g., such as by applying bandpass filters, changing a minimum or maximum volume, etc.) and services provided over the audio-based connection (e.g., such as, but not limited to, translation, content generation, automated communications, etc.

At block 204, the communication network may initialize the administrative session. The communication network may output an indication that the administrative session has been initiated. The indication may be phrase, such as “what administrative task would you like to complete today?” The indication may also be a tone, a beep, a song/jingle, a welcome message, any combination thereof, or the like.

At block 206, the communication manager may receive an audio segment from the first user device. The audio segment may include words spoken by the patient or a series of touch tones. For example, the first user device may transmit words or phrases, such as “schedule a new appointment,” “pay my bill,” “insurance,” etc. In some examples, the first user device may input one or more digits and/or symbols, generating a set of touch tones. The set of touch tones may be interpreted by the communication network as an audio segment.

At block 208, the communication manager may determine if the audio segment matches one or more system commands.

In some examples, a set of system commands may be stored in a database accessible by the communication network and/or the communication manager. Each command of the set of system commands may be stored in association with one or more executable functions, keywords/phrases associated with a command, related system commands, frequency of the command is invoked, any combination thereof, or the like. The communication manager may process an audio-segment received from the first user device to identify one or more natural language word or phrases (e.g., a speech-to-text model, etc.). The communication manager may execute classifier to identify one or more commands that correspond to the one or more natural language word or phrases. Alternatively, the communication manager may use the one or more natural language word or phrases to query the database to identify the one or more commands. The communication manager may also use a classifier to identify additional keywords/phrases with a same or similar semantic meaning as the one or more natural language word or phrases and use the additional keywords/phrases along with the one or more natural language word or phrases to query the database.

If a received communication includes two or more system commands, the communication manager may determine to execute commands in series or in parallel and an order in which to execute the two or more commands. In some instance the communication manager may schedule execution of each command of the two or more commands. If the system commands conflict, then both commands may be ignored. For example, the audio segment may contain instructions “cancel my next appointment and sign up for appointment reminders,” which may cause both commands to be ignored.

If a system command is detected that may require additional information and/or feedback from the first user device, the communication system may prompt the first user device. For example, the first user device may request to update a billing address and the communication network may, in response to the request, output an audio-segment including “what is your new address?”

At block 210, the communication network may execute the one or more system commands. Examples of system commands can include but are not limited to, updating a user profile, adding or modifying medical records, updating contact information, scheduling appointments, facilitating monetary transactions (e.g., via credit card, debit card, bank routing information, etc.), establishing an automated service (e.g., such as a bot or the like) configured to communicate with the user about the communication network and/or any communication sessions attended by the user, review electronic medical records, and the like. In some instances, a user (e.g., the patient, some associated with the patient, the healthcare providers, someone associated with the healthcare provider, etc.) may request access to information associated with the user stored by the communication. Since the audio-based connection may preclude visual representation of the information, the communication manager may use an automated service (e.g., such as a bot, etc.) or other machine-learning model to convert text-to-speech and perform content generation (e.g., large language model, etc.) to present the information using a synthetic voice over the audio-based connection. For non-text information (charts, graphs, etc.), a content generator may be used to describe the non-text information, provide explanation or qualifying information, etc. The user may ask questions to receive additional contextual information. For example, in response to presenting blood test results, the user may ask what a normal range for AIC levels are and receive a natural language response.

At block 212, the communication network may query the first user device, via a natural language audio communication, to determine whether the administrative session is complete. For example, after the communication network executes the system commands indicated by the first user device, the communication network may output an audio segment asking, “is that all you need today?” The first user device may respond with “yes” or “no” (e.g., transmitted as an audio segment, touch tones, etc.).

At block 214, if the first user device elects to conclude the administrative session, the communication network may terminate the administrative session causing the first user device to be disconnected from the administrative session.

After the administrative session terminates, the first user device may be automatically redirected to the top menu of the audio interface. In some examples, the communication network and/or the communication manager may automatically end the phone call.

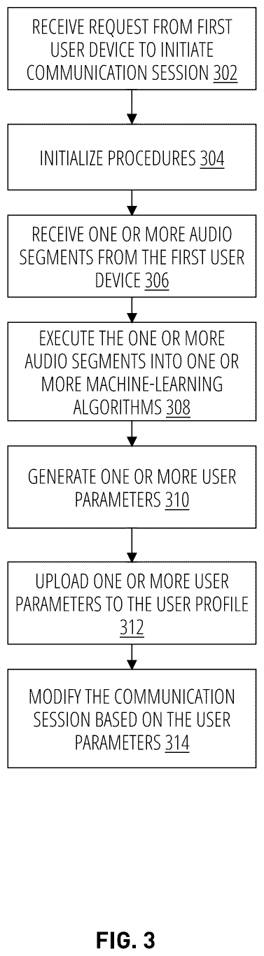

FIG. 3 illustrates a flowchart of an example process for initializing settings for a communication session according to audio input from the first user device according to aspects of the present disclosure. Although the example routine depicts a particular sequence of operations, the sequence may be altered without departing from the scope of the present disclosure. For example, some of the operations depicted may be performed in parallel or in a different sequence that does not materially affect the function of the routine. In other examples, different components of an example device or system that implements the routine may perform functions at substantially the same time or in a specific sequence.

At block 302, a computing device may receive a request from first user device to initiate communication session. The computing device may be a component of a communication network (e.g., such as communication network 120 of FIG. 1) and the first user device may be a user device operated by a patient. A communication session may be a session involving two or more user devices (one of which may be operated by an automated service configured for autonomous communications) that facilitates bidirectional communications over an audio-based communication channel (e.g., and/or one or more other communication channels text-based, audio-based, video-based, combinations thereof, or the like). For example, a communication session may be requested to establish a telehealth session between the patient and a healthcare provider operating a second user device. The communication session may be implemented using a communication manager (e.g., an application or process, etc.). The communication manager may be an interface enabling execution of one or more natural language machine-learning models (e.g., such as a large language model, bi-directional transformers, zero/few shot learners, deep neural networks, etc.) that provide additional functionality to the communication session. Alternatively, or additionally, the communication manager may include the one or more natural language machine-learning models. The one or more machine-learning models may be configured to process natural language communications (e.g., speech, inflection, language, etc.) and generate natural language responses that can be presented to the user devices of the communication session in a synthetic voice. In some examples, the responses may be audio (e.g., responding to a question from the first user device) or the responses may be task-based (e.g., updating the patient's home address).

The communication manager may be executed by the communication network such that when a user device connects to the communication network the communication manager may operate as an interface that can execute various functions of the communication network via commands received over the audio-based communication channel. Alternatively, or additionally, the communication manager may be executed on the user device (e.g., of the patient and/or the healthcare provider or any other party that may connect to communication sessions of the communication network). The communication manager may be configured to execute some functions of the communication network locally (on the user device) and, if needed, connect to the communication network for additional information (e.g., such as for electronic medical records, etc.). The communication manager executing on the user device may intercept communications to provide real time translations, provide definitions and/or other information, provide automated communication services such as bots, present and/or explain healthcare information (e.g., such as medical records, test results, etc.), replay communication sessions, filter communications, etc. In some instances, the communication manager may include a virtual assistant (e.g., a bot configured for the user that can be queried in natural language and provide natural language responses, etc.). For example, the user may present a wake word (e.g., an identifier of the virtual assistant), then provide a query that may be filtered from the audio-based communication channel to prevent the other user devices from hearing the query (e.g., using a bandpass filter or by temporarily muting communications transmitted by the users). The virtual assistant may then execute the requested command, provide natural language communications, etc.

In some examples, a second user device may be connected to the communication session. The second user device may be operated by a healthcare provider. In some examples, one or more additional user devices may access the communication session. For example, a clinician, a parent/guardian, and/or a child may also connect to the communication session. The second user device may be directly connected to the first user device (e.g., such as when the first user device calls the second user device over a telephone or voiceover Internet Protocol (VOIP) connection (or the like). In other instances, the first user device and the second user device may be connected through the communication network configured to manage the communication session and provide various functionality described herein (e.g., such as translation, access to databases, automated communications, etc.).

The user device may connect to the communication network to request a new communication session with the second user device through the communication manager. The communication network may query the first user device over the audio-based connection (e.g., using verbal prompts, etc.). For example, in response to the prompt, “what would you like to do today,” the patient may respond by stating “schedule an appointment with my doctor,” “ask a question about my medication,” “ask a question about my treatment plan,” “tell my doctor about a new symptom,” any combination thereof, or the like. The communication manager may use one or more machine-learning models to process verbal communications from the first user device. In some examples, the communication network may prompt the first user device to enter commands using touch tones.

The communication network may request additional input from the first user device. For example, the communication network may request from the first user device the timing preferred for the next appointment, a purpose of the communication session (e.g., checkup, new symptoms, etc.), what specific questions the patient may have (e.g., regarding the healthcare provider, treatment, medications, previous communication sessions, etc.). The communication manager may facilitate processing of the responses from the user to determine whether to connect the first user device to the second user device or to connect the first user device to an automated service (e.g., of the communication network or operating on a device associated with the healthcare provider). For example, the communication manager may classify an intent from responses from the first user device indicating the user has questions about a medication and may connect the first user device to an automated service configured to provide nature language communications (e.g., that simulate a human healthcare provider) regarding the medication. If the user is not satisfied with the communications with the automated service, the communication manager may establish a connection with a nurse, administrator, doctor, etc. to provide to communicate with the first user device.

At block 304, the computing device may execute initialization procedures that establish the communication session and configure the communication session (or the audio-based communication channel). The initialization procedures may include generating and initializing one or more parameters usable to configure the communication session. Some parameters may indicate when and how to establish the communication session (e.g., such as selected communication channels, an identification of user devices that will be connected, a date/time of the communication session, etc.). These parameters may be provided by user input, machine-learning models (e.g., based on inference, prediction, classification, etc.), algorithms, etc. Some parameters may be derived from a user profile associated with the first device. For example, the parameters may indicate an input or output volume of the communication session, a translation language for input audio-segments (e.g., received audio) and/or output audio-segments (e.g., transmitted audio). The parameters may be generated by one or more machine-learning models trained to recognize natural language and infer user preferences and/or settings (e.g., such as a large language model, bi-directional transformers, zero/few shot learners, deep neural networks, k-nearest neighbors, random forest, logarithmic regression, decision trees, support vector machines, autoencoder, gradient decent, etc.). For example, a machine-learning model may process input communications to determine what language the user is speaking.

If the communication manager determines that the first user device is to be connected with an automated communication service, the communication network may automatically initialize the communication session and configure the communication according to the parameters. For example, the communication manager may instantiate or execute one or more machine-learning models, functions, algorithms, other models, etc. that may process communications transmitted over the communication session in real time. If the communication manager determines that the communication session is to be with a healthcare provider, then the communication session may begin at the future date/time. The communication session may be initialized by the first user device by establishing a connection with the second user device over the selected communication channel (e.g., by calling the doctor at the scheduled time, dialing a phone number, clicking a link on an application and/or website, etc.). The communication manager may operate on the first user device and/or the second user device to provide the real time communication processing. In some examples, rather than wait until the future date/time, the communication manager may suspend the connection to the communication network (e.g., such as a hold, etc.) and establish the communication session with the second user device. In these examples, the initialization procedures may execute while the connection is suspended, prior to connecting the first user device and/or the second user device to the requested communication session. The initialization procedures may be executed and/or generated by the communication network, the communication manager, and/or a combination of the communication network and the communication manager.

At block 306, the computing device may receive one or more audio segments from the first user device over the communication session or the second user device. In some instances, the audio segment may be transmitted in response to a prompt or query transmitted to the user device that transmitted the audio segment. The audio segment may be a communication (e.g., natural language communication, a set of touch tones, etc.). For example, the communication network may ask “in what language are you most comfortable speaking with your healthcare provider” or “press two for Spanish.” The patient may respond with “Spanish,” or may press “two” to indicate Spanish, respectively. Alternatively, the communication manager may monitor the communication session and extract an audio segment transmitted by the first user device (e.g., such a communication spoken without a prompt to another user, etc.).

At block 308, the computing device may execute one or more machine-learning models using the audio segment. The communication manager may facilitate one or more machine-learning models trained to interpret natural language and classify semantics and/or context to infer user preferences and/or settings (e.g., such as a large language model, bi-directional transformers, zero/few shot learners, deep neural networks, k-nearest neighbors, random forest, logarithmic regression, decision trees, support vector machines, gradient decent, etc.).

For example, the communication manager may detect characteristics of a user based on the communications transmitted over the communication session. For example, the one or more machine-learning models may be configured (via the initialization procedure, or the like) to classify the language spoken by each user of the communication session (e.g., using speaker recognition to attribute the classified language to the user that communicated). The communication manager may detect a language, an accent (usable to query the user for a preferred language), a disability (e.g., such as hearing-impaired or hearing loss, etc.), and/or the like and modify the communication session based on the characteristics. The one or more machine-learning models may execute continuously throughout the communication session to detect changes in the languages spoken by the users. For example, if the communication network outputs a query and the user does not reply, the communication manager may determine that, based on the lack of a response, the user may have difficulty hearing, does not speak the language output, has poor network connectivity, etc.

The one or more machine-learning models may receive, in addition to the audio segment, data pertaining to the first user device (e.g., restrictions of the device, network connectivity, GPS location information, etc.), data pertaining to a user profile associated with the first user device, data from one or more additional user profiles, environmental data, any combination thereof, or the like.

At block 310, the computing device may generate one or more user parameters based on the output from the one or more machine-learning models. For example, the one or more machine-learning models may output an inference of a user characteristic (e.g., that the user speaks a particular language, etc.). The communication manager may then generate parameters based on the output characteristics. The generate parameters may include a preferred language, an output volume, a vocabulary assessment (indicating a degree of language competency or cognition, etc.), a recommend speech rate, any combination thereof, or the like.

At block 312, the computing device may upload the one or more user parameters to a user profile associated with the user. In some examples, the one or more user parameters may be usable to modify the current and subsequent communication sessions involving the user of the first user device. For example, in a subsequent communication session, the initialization procedures may be shorter or non-existent due to the stored user parameters. In some examples, the user parameters may be utilized to further train one or more machine-learning models associated with the communication network and/or the communication manager.

In some examples, the first user device may modify the one or more user parameters. For example, if a Spanish-speaking patient is now fluent in English, the patient may request at the top menu of the audio interface to update the user parameters. Subsequent communication sessions may reflect the updated user parameters.

At block 314, the computing device may modify the communication session based on the user parameters. The communication network, according to the user parameters, may update one or more communication session parameters associated with the communication session. The updated communication session parameters may include changing the output language, increasing/decreasing an output volume, including more/less frequent vocabulary definitions, slowing/increasing the pace of the output audio, providing translation or hearing-impairment assistance, any combination thereof, or the like. The communication manager may instantiate additional machine-learning models configured to facilitate any of the aforementioned modifications. For example, the communication manager may instantiate a machine-learning model configured to translate Spanish into English or connect an automated communication service to enable the first user device to request additional aid or receive additional explanations.

In some examples, the communication manager may continuously monitor and/or process communications transmitted over the communication session adjust the communication session (in real time). For example, over the duration of the communication session, the communication manager may detect that the user of the first user device is speaking English even though the initialization procedures configured to the communication session in Spanish and established a translation service for the first user device. The communication manger may then terminate the translation service.

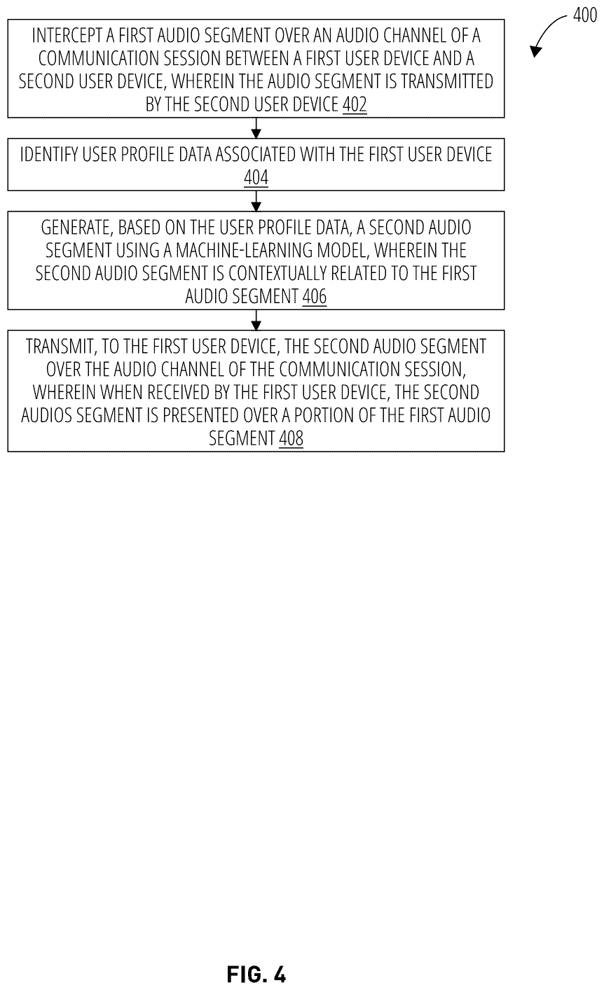

FIG. 4 illustrates a flowchart of an example process for facilitating a communication session between one or more user device on an audio communication platform. Although the example process depicts a particular sequence of operations, the sequence may be altered without departing from the scope of the present disclosure. For example, some of the operations depicted may be performed in parallel or in a different sequence that does not materially affect the function of the example process. In other examples, different components of an example device or system that implements the example process may perform functions at substantially the same time or in a specific sequence.

At block 402, a computing device may intercept a first audio segment over an audio channel of a communication session between a first user device and a second user device, wherein the audio segment may be transmitted by the second user device. The first user device may be operated by a patient and the second user device may be operated by a healthcare provider. In some instances, the computing device may be a component of a communication network. In other instances, the computing device may be the first user device or the second user device. The first audio segment may be extracted from an audio channel of the communication session. The audio segment may include natural language, dial tones combination thereof, or the like.

The communication session may be received by an audio interface configured to process communications and execute functions and/or services of the communication session. The audio interface may expose different functionality for each user device or class of user devices connected to the communication session. For example, the second user device may have access to one or more audio commands that the first user device may not have access to. The audio interface may enable enhanced presentation of information associated with the communication session during the communication session. For example, the communication session parameters may enable one or more machine-learning algorithms to supplement audio segments output by the second user device. For example, if the healthcare provider uses a medical term that is typically unfamiliar to the average patient, the one or more machine-learning algorithms may output a definition of the medical term to the patient via a second audio segment.

At block 404, the computing device may identify user profile data associated with the first user device. User devices connected to the communication session may be associated with individual user profiles stored within, or in association with, the computing device. The individual user profiles may be completed and stored prior to the communication session. The user profile data within the individual user profiles may include, but is not limited to, one or more user parameters (e.g., the user parameters described in FIG. 3), demographic data (e.g., race, gender, ethnicity, etc.), health and medical history (e.g., height, weight, past surgeries, etc.), preferences (e.g., prefers injections over oral medications), allergies, familial status, contact information, insurance information, data associated with prior communication sessions connected to by the user device associated with the individual user profile, healthcare provider information (e.g., primary care physician, OB/GYN, physical therapist, etc.), medical practice history, lifestyle data (e.g., smoking and alcohol frequency, exercise frequency, tendency to remember to take daily medication, tendency to cancel/reschedule/no-show communication sessions and/or other healthcare appointments, stress level, pollution level of living environment, cleanliness, eating habits, etc.), any combination thereof, or the like.

At block 406, the computing device may generate, based on the user profile data, a second audio segment using a machine-learning model, wherein the second audio segment is contextually related to the first audio segment. In some examples, the first audio segment may be interpreted by a machine-learning algorithm, natural language processing machine-learning model, etc. by converting the communication into a neutral format (e.g., alphanumeric text using one or more machine-learning model such as a speech-to-text model, etc.) and classifying the neutral format (e.g., using the one or more machine-learning models or other machine-learning models). In other instances, the first audio segment may be interpreted without first converting the communication into a neutral format. For example, a first machine-learning model may receive the first audio segment and generate one or more modifications, thereby generating the second audio segment. In some examples, no modifications may be necessary, and the first audio segment may be substantially similar to the second audio segment. In some examples, the machine-learning model may be configured to generate new audio segment from scratch.

The modifications may include a translation (e.g., first audio segment includes a set of words spoken by a user of the second user device in a first language, and wherein the second audio segment includes a translation of the set of words in a second language or a version of the audio segment that may be easier for a hearing impaired or foreign user to understand), a modified waveform (e.g., such as a version of the first audio segment with a higher or lower volume, certain frequencies filtered, etc.), a slower/faster pace of output (e.g., the second audio segment is configured to be output at a slower pace than the first audio segment, thereby delivering the same information over a longer duration of time and enabling increased comprehension for the patient), vocabulary definitions (e.g., the second audio segment includes a definition of a word included in the first audio segment), any combination thereof, or the like.

At block 408, the computing device may transmit, to the first user device, the second audio segment over the audio channel of the communication session, wherein when received by the first user device, the second audio segment may be presented. The second audio segment may be presented over the first audio segment (e.g., synchronized, or non-synonymized with the first audio segment) or offset from the first audio segment (e.g., after the first audio segment terminates). The first and second audio segments may be presented according to a presentation pattern generated by one or more machine-learning models trained to interpret natural language and infer user preferences and/or settings. For example, a second machine-learning model may generate a presentation pattern applicable to the first and second audio segment according to data associated with a user profile of the patient so as to tailor presentation of the second audio segment to the user.

In some examples, the second audio segment may be presented in lieu of the first audio segment. In other examples, the second audio segment may supplement the first audio segment according to the presentation pattern. For example, the first user device may present a portion of the first audio segment and a portion of the second audio segment. The presentation pattern may optimize the contents of the first audio segment and the second audio segment to present the context of the first audio segment to the first user device. The optimization may be based upon brevity, conciseness, clarity, thoroughness, and/or any preference inferred from the associated user profile, user parameters, etc.

In some examples, a filter may be used to prevent the second audio segment from being received by the second user device or other user devices connected to the communication session. The filter may override the presentation pattern associated with the first audio segment and the second audio segment. For example, the user of the first user device an enable or disable the filter to selectively enable receiving machine-learning-generated output and/or modifications. In some instance, the second user device may enable or disable the filter or any machine-learning-generated output and/or modifications to the communication channel on behalf of the first user device and/or the user thereof.

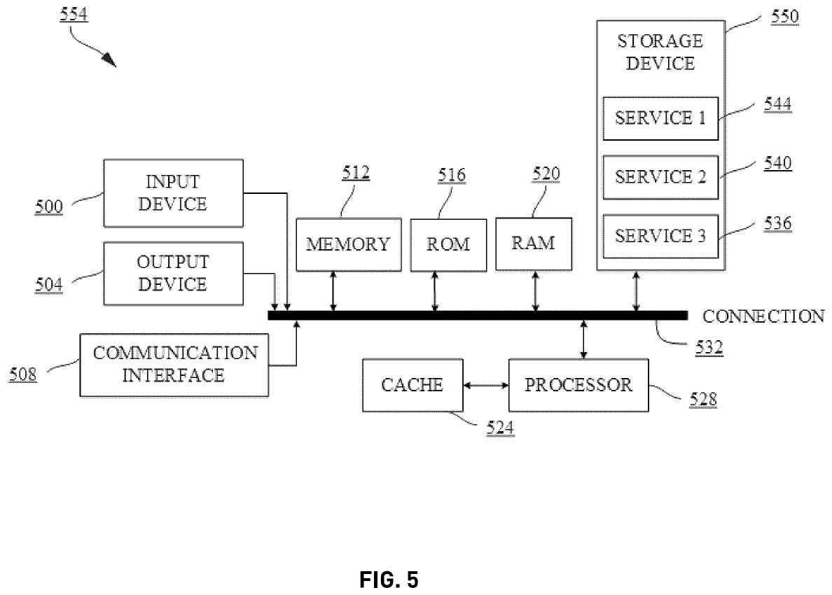

FIG. 5 illustrates an example computing device according to aspects of the present disclosure. For example, computing device 500 can implement any of the systems or methods described herein. In some instances, computing device 500 may be a component of or included within a media device. The components of computing device 500 are shown in electrical communication with each other using connection 506, such as a bus. The example computing device architecture 500 includes a processor (e.g., CPU, processor, or the like) 504 and connection 506 (e.g., such as a bus, or the like) that is configured to couple components of computing device 500 such as, but not limited to, memory 520, read only memory (ROM) 518, random access memory (RAM) 516, and/or storage device 508, to processing unit 510.

Computing device 500 can include a cache 502 of high-speed memory connected directly with, in close proximity to, or integrated within processor 504. Computing device 500 can copy data from memory 520 and/or storage device 508 to cache 502 for quicker access by processor 504. In this way, cache 502 may provide a performance boost that avoids delays while processor 504 waits for data. Alternatively, processor 504 may access data directly from memory 520, ROM 517, RAM 516, and/or storage device 508. Memory 520 can include multiple types of homogenous or heterogeneous memory (e.g., such as, but not limited to, magnetic, optical, solid-state, etc.).

Storage device 508 may include one or more non-transitory computer-readable media such as volatile and/or non-volatile memories. A non-transitory computer-readable medium can store instructions and/or data accessible by computing device 500. Non-transitory computer-readable media can include, but is not limited to magnetic cassettes, hard-disk drives (HDD), flash memory, solid state memory devices, digital versatile disks, cartridges, compact discs, random access memories (RAMs) 525, read only memory (ROM) 520, combinations thereof, or the like.

Storage device 508, may store one or more services, such as service 1 510, service 2 512, and service 3 514, that are executable by processor 504 and/or other electronic hardware. The one or more services include instructions executable by processor 504 to: perform operations such as any of the techniques, steps, processes, blocks, and/or operations described herein; control the operations of a device in communication with computing device 500; control the operations of processing unit 510 and/or any special-purpose processors; combinations therefor; or the like. Processor 504 may be a system on a chip (SOC) that includes one or more cores or processors, a bus, memories, clock, memory controller, cache, other processor components, and/or the like. A multi-core processor may be symmetric or asymmetric.

Computing device 500 may include one or more input devices 522 that may represent any number of input mechanisms, such as a microphone, a touch-sensitive screen for graphical input, keyboard, mouse, motion input, speech, media devices, sensors, combinations thereof, or the like. Computing device 500 may include one or more output devices 524 that output data to a user. Such output devices 524 may include, but are not limited to, a media device, projector, television, speakers, combinations thereof, or the like. In some instances, multimodal computing devices can enable a user to provide multiple types of input to communicate with computing device 500. Communications interface 526 may be configured to manage user input and computing device output. Communications interface 526 may also be configured to managing communications with remote devices (e.g., establishing connection, receiving/transmitting communications, etc.) over one or more communication protocols and/or over one or more communication media (e.g., wired, wireless, etc.).

Computing device 500 is not limited to the components as shown in FIG. 5. Computing device 500 may include other components not shown and/or components shown may be omitted.

The term “computer-readable medium” includes, but is not limited to, portable or non-portable storage devices, optical storage devices, and various other mediums capable of storing, containing, or carrying instruction(s) and/or data. A computer-readable medium may include a non-transitory medium in which data can be stored in a form that excludes carrier waves and/or electronic signals. Examples of a non-transitory medium may include, but are not limited to, a magnetic disk or tape, optical storage media such as compact disk (CD) or digital versatile disk (DVD), flash memory, memory, or memory devices. A computer-readable medium may have stored thereon code and/or machine-executable instructions that may represent a procedure, a function, a subprogram, a program, a routine, a subroutine, a module, a software package, a class, or any combination of instructions, data structures, or program statements. A code segment may be coupled to another code segment or a hardware circuit by passing and/or receiving information, data, arguments, parameters, or memory contents. Information, arguments, parameters, data, etc. may be passed, forwarded, or transmitted via any suitable means including memory sharing, message passing, token passing, network transmission, or the like.

As used below, any reference to a series of examples is to be understood as a reference to each of those examples disjunctively (e.g., “Examples 1-4” is to be understood as “Examples 1, 2, 3, or 4”).

Example 1 is a method comprising: intercepting a first audio segment over an audio channel of a communication session between a first user device and a second user device, wherein the first audio segment is transmitted by the second user device; identifying user profile data associated with the first user device; generating, based on the user profile data, a second audio segment using a machine-learning model, wherein the second audio segment is contextually related to the first audio segment; and transmitting, to the first user device, the second audio segment over the audio channel of the communication session, wherein when received by the first user device, the second audio segment is presented over a portion of the first audio segment.

Example 2 is the method of example(s) 1, where in the second audio segment is configured to be presented at an offset from the first audio segment.

Example 3 is the method of example(s) 1-2, where in the second audio segment is configured to be presented at a different volume than the first audio segment.

Example 4 is the method of example(s) 1-3, wherein the first audio segment includes a set of words spoken by a user of the second user device in a first language, and wherein the second audio segment includes a translation of the set of words in a second language.

Example 5 is the method of example(s) 1-4, wherein the second audio segment includes a definition of a word included in the first audio segment.

Example 6 is the method of example(s) 1-5, wherein the second audio segment includes an explanation of a word or phrase included in the first audio segment.

Example 7 is the method of example(s) 1-6, wherein a filter of the audio channel prevents the second audio segment from being received by the second user device.

Example 8 is a system comprising of one or more processor and a non-transitory computer-readable medium storing instructions that when executed by the one or more processors cause the one or more processors to perform the methods of any of example(s) 1-7.

Example 9 is a non-transitory computer-readable medium storing instructions that when executed by the one or more processors cause the one or more processors to perform the methods of any of example(s) 1-7.

Some portions of this description describe examples in terms of algorithms and symbolic representations of operations on information. These operations, while described functionally, computationally, or logically, may be implemented by computer programs or equivalent electrical circuits, microcode, or the like. Furthermore, arrangements of operations may be referred to as modules, without loss of generality. The described operations and their associated modules may be embodied in software, firmware, hardware, or any combinations thereof.

Any of the steps, operations, or processes described herein may be performed or implemented with one or more hardware or software modules, alone or in combination with other devices. In some examples, a software module can be implemented with a computer-readable medium storing computer program code, which can be executed by a processor for performing any or all of the steps, operations, or processes described.

Some examples may relate to an apparatus or system for performing any or all of the steps, operations, or processes described. The apparatus or system may be specially constructed for the required purposes, and/or it may comprise a general-purpose computing device selectively activated or reconfigured by a computer program stored in memory of computing device. The memory may be or include a non-transitory, tangible computer readable storage medium, or any type of media suitable for storing electronic instructions, which may be coupled to a bus. Furthermore, any computing systems referred to in the specification may include a single processor or multiple processors.

While the present subject matter has been described in detail with respect to specific examples, it will be appreciated that those skilled in the art, upon attaining an understanding of the foregoing, may readily produce alterations to, variations of, and equivalents to such embodiments. Numerous specific details are set forth herein to provide a thorough understanding of the claimed subject matter. However, those skilled in the art will understand that the claimed subject matter may be practiced without these specific details. In other instances, methods, apparatuses, or systems that would be known by one of ordinary skill have not been described in detail so as not to obscure claimed subject matter. Accordingly, the present disclosure has been presented for purposes of example rather than limitation, and does not preclude the inclusion of such modifications, variations, and/or additions to the present subject matter as would be readily apparent to one of ordinary skill in the art.

For clarity of explanation, in some instances the present disclosure may be presented as including individual functional blocks including functional blocks comprising devices, device components, steps or routines in a method embodied in software, or combinations of hardware and software. Additional functional blocks may be used other than those shown in the figures and/or described herein. For example, circuits, systems, networks, processes, and other components may be shown as components in block diagram form in order not to obscure the embodiments in unnecessary detail. In other instances, well-known circuits, processes, algorithms, structures, and techniques may be shown without unnecessary detail in order to avoid obscuring the embodiments.

Individual examples may be described herein as a process or method which may be depicted as a flowchart, a flow diagram, a data flow diagram, a structure diagram, or a block diagram. Although a flowchart may describe the operations as a sequential process, many of the operations can be performed in parallel or concurrently. In addition, the order of the operations may be re-arranged. A process is terminated when its operations are completed but may have additional steps not shown. A process may correspond to a method, a function, a procedure, a subroutine, a subprogram, etc. When a process corresponds to a function, its termination can correspond to a return of the function to the calling function or the main function.

Processes and methods according to the above-described examples can be implemented using computer-executable instructions that are stored or otherwise available from computer-readable media. Such instructions can include, for example, instructions and data which cause or otherwise configure a general-purpose computer, special purpose computer, or a processing device to perform a certain function or group of functions. Portions of computer resources used can be accessible over a network. The computer executable instructions may be, for example, binaries, intermediate format instructions such as assembly language, firmware, source code, etc.

Devices implementing the methods and systems described herein can include hardware, software, firmware, middleware, microcode, hardware description languages, or any combination thereof, and can take any of a variety of form factors. When implemented in software, firmware, middleware, or microcode, the program code or code segments to perform the necessary tasks (e.g., a computer-program product) may be stored in a computer-readable or machine-readable medium. The program code may be executed by a processor, which may include one or more processors, such as, but not limited to, one or more digital signal processors (DSPs), general purpose microprocessors, an application specific integrated circuits (ASICs), field programmable logic arrays (FPGAs), or other equivalent integrated or discrete logic circuitry. Such a processor may be configured to perform any of the techniques described in this disclosure. A processor may be a microprocessor; conventional processor, controller, microcontroller, state machine, or the like. A processor may also be implemented as a combination of computing components (e.g., a combination of a DSP and a microprocessor, a plurality of microprocessors, one or more microprocessors in conjunction with a DSP core, or any other such configuration). Accordingly, the term “processor,” as used herein may refer to any of the foregoing structure, any combination of the foregoing structure, or any other structure or apparatus suitable for implementation of the techniques described herein. Functionality described herein also can be embodied in peripherals or add-in cards. Such functionality can also be implemented on a circuit board among different chips or different processes executing in a single device, by way of further example.

In the foregoing description, aspects of the disclosure are described with reference to specific examples thereof, but those skilled in the art will recognize that the disclosure is not limited thereto. Thus, while illustrative examples of the disclosure have been described in detail herein, it is to be understood that the inventive concepts may be otherwise variously embodied and employed, and that the appended claims are intended to be construed to include such variations. Various features and aspects of the above-described disclosure may be used individually or in any combination. Further, examples can be utilized in any number of environments and applications beyond those described herein without departing from the broader spirit and scope of the disclosure. The disclosure and figures are, accordingly, to be regarded as illustrative rather than restrictive.

The various illustrative logical blocks, modules, circuits, and algorithm steps described in connection with the embodiments disclosed herein may be implemented as electronic hardware, computer software, firmware, or combinations thereof. To clearly illustrate this interchangeability of hardware and software, various illustrative components, blocks, modules, circuits, and steps have been described above generally in terms of their functionality. Whether such functionality is implemented as hardware or software depends upon the particular application and design constraints imposed on the overall system. Skilled artisans may implement the described functionality in varying ways for each particular application, but such implementation decisions should not be interpreted as causing a departure from the scope of the present application.

Unless specifically stated otherwise, it is appreciated that throughout this specification discussions utilizing terms such as “processing,” “computing,” “calculating,” “determining,” and “identifying” or the like refer to actions or processes of a computing device, such as one or more computers or a similar electronic computing device or devices, that manipulate or transform data represented as physical electronic or magnetic quantities within memories, registers, or other information storage devices, transmission devices, or media devices of the computing platform. The use of “adapted to” or “configured to” herein is meant as open and inclusive language that does not foreclose devices adapted to or configured to perform additional tasks or steps. Additionally, the use of “based on” is meant to be open and inclusive, in that a process, step, calculation, or other action “based on” one or more recited conditions or values may, in practice, be based on additional conditions or values beyond those recited. Headings, lists, and numbering included herein are for ease of explanation only and are not meant to be limiting.

The foregoing detailed description of the technology has been presented for purposes of illustration and description. It is not intended to be exhaustive or to limit the technology to the precise form disclosed. Many modifications and variations are possible in light of the above teaching. The described embodiments were chosen in order to best explain the principles of the technology, its practical application, and to enable others skilled in the art to utilize the technology in various embodiments and with various modifications as are suited to the particular use contemplated. It is intended that the scope of the technology be defined by the claim.

What is claimed is:

1. A computer-implemented method comprising:
intercepting a first audio segment over an audio channel of a communication session between a first user device and a second user device, wherein the first audio segment is transmitted by the second user device;
identifying user profile data associated with the first user device;
generating, based on the user profile data, a second audio segment using a machine-learning model, wherein the second audio segment is contextually related to the first audio segment; and
transmitting, to the first user device, the second audio segment over the audio channel of the communication session, wherein when received by the first user device, the second audio segment is presented over a portion of the first audio segment.

2. The computer-implemented method of claim 1, where in the second audio segment is configured to be presented at an offset from the first audio segment.

3. The computer-implemented method of claim 1, where in the second audio segment is configured to be presented at a different volume than the first audio segment.

4. The computer-implemented method of claim 1, wherein the first audio segment includes a set of words spoken by a user of the second user device in a first language, and wherein the second audio segment includes a translation of the set of words in a second language.

5. The computer-implemented method of claim 1, wherein the second audio segment includes a definition of a word included in the first audio segment.

6. The computer-implemented method of claim 1, wherein the second audio segment includes an explanation of a word or phrase included in the first audio segment.

7. The computer-implemented method of claim 1, wherein a filter of the audio channel prevents the second audio segment from being received by the second user device.

8. A system comprising:
one or more processors and a non-transitory computer-readable medium storing instructions that when executed by the one or more processors cause the one or more processors to perform operations that include:
intercepting a first audio segment over an audio channel of a communication session between a first user device and a second user device, wherein the first audio segment is transmitted by the second user device;
identifying user profile data associated with the first user device;
generating, based on the user profile data, a second audio segment using a machine-learning model, wherein the second audio segment is contextually related to the first audio segment; and
transmitting, to the first user device, the second audio segment over the audio channel of the communication session, wherein when received by the first user device, the second audio segment is presented over a portion of the first audio segment.

9. The system of claim 8, where in the second audio segment is configured to be presented at an offset from the first audio segment.

10. The system of claim 8, where in the second audio segment is configured to be presented at a different volume than the first audio segment.

11. The system of claim 8, wherein the first audio segment includes a set of words spoken by a user of the second user device in a first language, and wherein the second audio segment includes a translation of the set of words in a second language.

12. The system of claim 8, wherein the second audio segment includes a definition of a word included in the first audio segment.

13. The system of claim 8, wherein the second audio segment includes an explanation of a word or phrase included in the first audio segment.

14. The system of claim 8, wherein a filter of the audio channel prevents the second audio segment from being received by the second user device.

15. A non-transitory computer-readable medium storing instructions that when executed by one or more processors cause the one or more processors to perform operations that include:
intercepting a first audio segment over an audio channel of a communication session between a first user device and a second user device, wherein the first audio segment is transmitted by the second user device;
identifying user profile data associated with the first user device;
generating, based on the user profile data, a second audio segment using a machine-learning model, wherein the second audio segment is contextually related to the first audio segment; and
transmitting, to the first user device, the second audio segment over the audio channel of the communication session, wherein when received by the first user device, the second audio segment is presented over a portion of the first audio segment.

16. The non-transitory computer-readable medium of claim 15, where in the second audio segment is configured to be presented at an offset from the first audio segment.

17. The non-transitory computer-readable medium of claim 15, where in the second audio segment is configured to be presented at a different volume than the first audio segment.

18. The non-transitory computer-readable medium of claim 15, wherein the first audio segment includes a set of words spoken by a user of the second user device in a first language, and wherein the second audio segment includes a translation of the set of words in a second language.

19. The non-transitory computer-readable medium of claim 15, wherein the second audio segment includes a definition of a word included in the first audio segment.

20. The non-transitory computer-readable medium of claim 15, wherein the second audio segment includes an explanation of a word or phrase included in the first audio segment.
